(19) United States
(12) Patent Application Publication
(10) Pub. No.: US 2026/0034138 A1
(43) Pub. Date: Feb. 5, 2026

(54) NOVEL PYRIMIDINYL AND TRIAZINYL SULFONAMIDE DERIVATIVES

(71) Applicant: Hoffmann-La Roche Inc., Little Falls, NJ (US)

(72) Inventors: Guido GALLEY, Rheinfelden (DE); Luca GOBBI, Buus (CH); Wolfgang GUBA, Muellheim (DE); Dmitry MAZUNIN, Grenzach-Wyhlen (DE); Emmanuel PINARD, Linsdorf (FR); Antonio RICCI, Biel-Benken (CH)

(73) Assignee: Hoffmann-La Roche Inc., Little Falls, NJ (US)

(21) Appl. No.: 18/994,499

(22) PCT Filed: Jul. 18, 2023
(86) PCT No.: PCT/EP2023/069859

§ 371 Date: Jan. 14, 2025

(30) Foreign Application Priority Data
Jul. 20, 2022 (EP) 22185886.3
Aug. 2, 2022 (EP) 22188205.3

(51) Int. Cl. A61K 31/53; A61K 31/506; C07D 403/12; C07D 471/04

(52) U.S. Cl. CPC A61K 31/53; A61K 31/506; C07D 403/12; C07D 471/04

(57) ABSTRACT

The invention relates to novel compounds having the general formula (I) wherein R⁴, R⁵, X₁, X₂, X₃ and Y₁ are as described herein, composition including the compounds and methods of using the compounds.

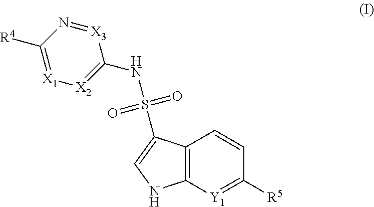

The present invention relates to organic compounds useful for therapy and/or prophylaxis in a mammal, and in particular to compounds that modulate GPR17 activity.

The present invention provides novel compounds of formula I

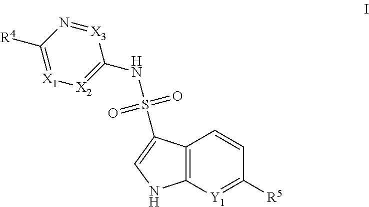

wherein,
R⁴ is alkyl, halo, haloalkyl, or haloalkoxy;
R⁵ is halo or haloalkyl;
X₁ is N, X₂ is CR² and X₃ is CR³, wherein R² is H and R³ is alkoxy, halo, or haloalkoxy,
or
X₁ is CR¹, X₂ is N and X₃ is N, wherein R¹ is alkoxy;
Y₁ is CR⁶ or N;
R⁶ is H or halo;
and pharmaceutically acceptable salts thereof.

Furthermore, the invention includes all racemic mixtures, all their corresponding enantiomers and/or optical isomers.

### BACKGROUND OF THE INVENTION

Myelination is a process that occurs robustly during development and despite the abundant presence of oligodendrocyte precursor cells (OPCs) throughout the adult CNS, the transition to myelinating oligodendrocytes and the production of restorative myelin sheaths around denuded axons is impaired in chronic demyelinating diseases. During development, myelination proceeds in a very orderly manner, with OPCs, characterized by expression of markers such as neural/glial antigen 2 (NG2) and platelet-derived growth factor alpha (PDGFRα), differentiating into oligodendrocytes which lose NG2 and PDGFRα expression and gain the expression of markers such as myelin basic protein (MBP) and myelin oligodendrocyte glycoprotein (MOG). The production of myelin by oligodendrocytes is a very tightly regulated process and in the CNS, this can be controlled by interactions with axons, well-understood in the peripheral but not in the central nervous system (Macklin, W. B. (2010). Sci. Signal. 3, pe32-pe32, “The myelin brake: When Enough Is Enough”). Myelination can also be controlled by internal brakes within oligodendrocytes themselves, through the transcription factor EB (TFEB)-PUMA axis or through GPR17 antagonism (Chen, Y., et al. (2009). Nat Neurosci 12, 1398-1406, “The oligodendrocyte-specific G protein-coupled receptor GPR17 is a cell-intrinsic timer of myelination”) (Sun, L. O., et al. (2018). Cell 175, 1811-1826.e21, “Spatiotemporal Control of CNS Myelination by Oligodendrocyte Programmed Cell Death through the TFEB-PUMA Axis”). Myelin serves not only to protect axons and facilitate neuronal transmission, but oligodendrocytes have also been shown to play an important role in metabolism of axons as well as in maintaining the electrolyte balance around axons (Schirmer, L., et al. (2014). Ann Neurol 75, 810-828, “Differential loss of KIR4.1 immunoreactivity in multiple sclerosis lesions”) (Simons, M., and Nave, K.-A. (2015). Cold Spring Harb Perspect Biol. 22, “Oligodendrocytes: Myelination and Axonal Support”).

GPR17 is a Class A orphan G protein-coupled receptor (GPCR). GPCRs are 7 domain transmembrane proteins that couple extracellular ligands with intracellular signaling via their intracellular association with small, heterotrimeric G-protein complexes consisting of G_{α}, G_{β}, G_{γ} subunits. It is the coupling of the GPCR to the G_{α} subunit that confers results in downstream intracellular signaling pathways. GPR17 is known to be coupled directly to G_{α i/o}, which leads to inhibition of adenylate cyclase activity, resulting in a reduction in cyclic AMP production (cAMP). GPR17 has also been shown to couple to G_{q/11}, that targets phospholipase C. Activation of phospholipase C leads to the cleavage of phosphatidylinositol 4,5-bisphosphate which produces inositol triphosphate (IP₃) and diacylglycerol (DAG). IP₃ consequently binds to the IP₃ receptor on the endoplasmic reticulum and causes an increase in intracellular calcium levels (Hanlon, C. D., and Andrew, D. J. (2015). J Cell Sci. 128, 3533-3542, “Outside-in signaling—a brief review of GPCR signaling with a focus on the Drosophila GPCR family”) (Inoue, A., et al. (2019), Cell 177, 1933-1947.e25, “Illuminating G-Protein-Coupling Selectivity of GPCRs”).

The role of GPR17 in myelination was first identified in a screen of the optic nerves of Olig1 knockout mice to identify genes regulating myelination. GPR17 expression was found to be expressed only in the myelinating cells of the CNS and absent from the Schwann cells, the peripheral nervous system's myelinating cells. The expression of GPR17 was found to be exclusively expressed in the oligodendrocyte lineage cells and was downregulated in myelinating oligodendrocyte (Chen, Y., et al. (2009)). Specifically, GPR17 expression is found to be present at low levels early on in the OPC and increases in the pre-myelinating oligodendrocyte before the expression is downregulated in the mature, myelinating oligodendrocyte (Boda, E., et al. (2011), Glia 59, 1958-1973, “The GPR17 receptor in NG2 expressing cells: Focus on in vivocell maturation and participation in acute trauma and chronic damage”) (Dziedzic, A., et al. (2020). Int. J. Mol. Sci. 21, 1852, “The gpr17 receptor—a promising goal for therapy and a potential marker of the neurodegenerative process in multiple sclerosis”) (Fumagalli, M. et al. (2011), J Biol Chem 286, 10593-10604, “Phenotypic changes, signaling pathway, and functional correlates of GPR17-expressing neural precursor cells during oligodendrocyte differentiation”). GPR17 knockout animals were shown to exhibit precocious myelination throughout the CNS and conversely, transgenic mice overexpressing GPR17 in oligodendrocytes with the CNP-Cre (2′,3′-cyclic-nucleotide 3′-phosphodiesterase) promoter exhibited myelinogenesis defects, in line with what is to be expected of a cell-intrinsic brake on the myelination process (Chen, Y., et al. (2009)). Furthermore, loss of GPR17 enhances remyelination following demyelination with lysophosphatidylcholine-induced demyelination (Lu, C., Dong, et al. (2018), Sci. Rep. 8, 4502, “G-Protein-Coupled Receptor Gpr17 Regulates Oligodendrocyte Differentiation in Response to Lysolecithin-Induced Demyelination”). As such, antagonism of GPR17 that promotes the differentiation of oligodendrocyte lineage cells into mature, myelinating oligodendrocytes would lead to increase in myelination following demyelination.

Multiple sclerosis (MS) is a chronic neurodegenerative disease that is characterized by the loss of myelin, the protective fatty lipid layer surrounding axons, in the central nervous system (CNS). Prevention of myelin loss or remyelination of denuded axons is thought to prevent axonal degeneration and thus prevent progression of the disease (Franklin, R. J. (2002), Nat Rev Neurosci 3, 705-714, “Why does remyelination fail in multiple sclerosis?”). Due to the restorative impact that myelin repair has on the central nervous system, such a treatment will benefit all types of MS namely relapse-remitting, secondary progressive, primary progressive and progressive relapsing MS. Reparation of lost myelin will alleviate neurological symptoms associated with MS due to the neuroprotective effect of preserving axons.

Due to the essential role that myelination plays in functioning of the nervous system, facilitating OPC to oligodendrocyte differentiation has the potential to impact multiple diseases where white matter defects/irregularities due to either loss of myelinating oligodendrocytes or hampered differentiation of OPCs to oligodendrocytes have been observed, due to the disease itself or inflammation. This is in addition to the diseases where GPR17 expression itself is altered.

The diseases that GPR17 antagonism can be thus used to yield a positive disease outcome include, but are not limited to:

### Direct Damage to Myelin Sheaths:

Metabolic conditions that lead to destruction of central myelin such as central pontine myelinolysis, extra-pontine myelinolysis due to overly-rapid correction of hyponatremia in conditions for instance, but not limited to, alcoholism, liver disease, immunosuppression after transplantation
Carbon monoxide poisoning where oligodendrocyte dysfunction and failure to regenerate has been reported in the deep white matter layers of the brain
Nutritional deficiency that results in myelin loss or failure to properly generate myelin during development
Virus-induced demyelination

### Primary Demyelinating Disorders

Multiple Sclerosis (relapse-remitting, secondary progressive, primary progressive and progressive relapsing MS)
Acute and multiphasic disseminated encephalomyelitis
Neuromyelitis optica spectrum disorders including optic neuritis
Transverse myelitis
Leukodystrophies such as adrenoleukodystrophy, adrenomyeloneuropathy and other inherited leukodystrophies that result in myelin loss
   CNS Disorders with Associated Myelin Loss:
Alzheimer's Disease
Schizophrenia
Parkinson's Disease
Huntington's disease
Amyotrophic lateral
Ischemia due to stroke

### Other Diseases:

Inflammation in the CNS for instance following encephalitis, primary angiitis, meningitis

The compounds of formula I bind to and modulates GPR17 activity.

The compounds of formula I are therefore particularly useful in the treatment of diseases related to GPR17 antagonism.

The compounds of formula I are particularly useful in the treatment or prophylaxis of multiple sclerosis (MS), conditions related to direct damage to myelin sheaths such as carbon monoxide poisoning or virus induced demyelination, primary demyelinating disorders such as acute and multiphasic disseminated encephalomyelitis, and other CNS disorders associated with myelin loss such as Alzheimer's disease, schizophrenia, Parkinson's disease and Huntington's disease.

### SUMMARY OF THE INVENTION

The present invention provides novel compounds of formula I

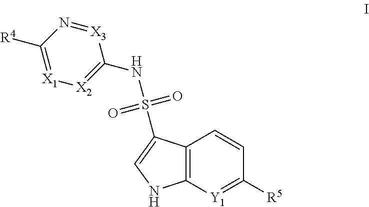

wherein,
R⁴ is alkyl, halo, haloalkyl, or haloalkoxy;
R⁵ is halo or haloalkyl;
X₁ is N, X₂ is CR² and X₃ is CR³, wherein R² is H and R³ is alkoxy, halo, or haloalkoxy,
or
X₁ is CR¹, X₂ is N and X₃ is N, wherein R¹ is alkoxy;
Y₁ is CR⁶ or N;
R⁶ is H or halo;
and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl, ethyl and propyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy. Particular examples are methoxy and ethoxy.

The term “halogen”, “halide” and “halo” are used interchangeably herein and denote fluoro, chloro, bromo or iodo. Particular halogens are fluoro, chloro and bromo.

The term “haloalkyl” denotes a C₁₋₆-alkyl group wherein at least one of the hydrogen atoms of the C₁₋₆-alkyl group has been replaced by the same or different halogen atoms. Particular examples are difluoroethyl and trifluoromethyl.

The term “haloalkoxy” denotes a C₁₋₆-alkoxy group wherein at least one of the hydrogen atoms of the C₁₋₆-alkoxy group has been replaced by the same or different halogen atoms. Particular examples fluoroethoxy, difluoromethoxy, and difluoroethoxy.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R⁴ is haloalkoxy.

An embodiment of the present invention provides compounds according to formula I as described herein, wherein R⁵ is halo.

An embodiment of the present invention provides compounds according to formula I as described herein, wherein R⁶ is H.

An embodiment of the present invention provides compounds according to formula I as described herein, wherein X₁ is N, X₂ is CR², and X₃ is CR³ wherein R² is H and R³ is alkoxy, or haloalkoxy.

An embodiment of the present invention provides compounds according to formula I as described herein, wherein
R⁴ is haloalkoxy;
R⁵ is halo;
X₁ is N, X₂ is CR² and X₃ is CR³, wherein R² is H and R³ is alkoxy or haloalkoxy;
Y₁ is CR⁶ or N;
R⁶ is H;
and pharmaceutically acceptable salts thereof.

Particular examples of compounds of formula I as described herein are selected from
6-chloro-N-(4-methoxy-2-propyl-pyrimidin-5-yl)-1H-indole-3-sulfonamide;
6-chloro-N-(2-ethyl-4-methoxy-pyrimidin-5-yl)-1H-indole-3-sulfonamide;
6-chloro-N-[2-(difluoromethoxy)-4-methoxy-pyrimidin-5-yl]-1H-indole-3-sulfonamide;
6-chloro-N-[2-(2,2-difluoroethoxy)-4-methoxy-pyrimidin-5-yl]-1H-pyrrolo[2,3-b]pyridine-3-sulfonamide;
6-chloro-N-[2-(2,2-difluoroethoxy)-4-(difluoromethoxy)pyrimidin-5-yl]-1H-indole-3-sulfonamide;
6-chloro-N-(2,4-dichloropyrimidin-5-yl)-1H-indole-3-sulfonamide;
6-chloro-N-(2-(2-fluoroethoxy)-4-methoxypyrimidin-5-yl)-1H-indole-3-sulfonamide;
6-chloro-N-(2-(2,2-difluoroethoxy)-4-methoxypyrimidin-5-yl)-1H-indole-3-sulfonamide;
6-bromo-N-[2-(2,2-difluoroethoxy)-4-methoxy-pyrimidin-5-yl]-1H-indole-3-sulfonamide;
6-bromo-N-[2-(2,2-difluoroethoxy)-4-methoxy-pyrimidin-5-yl]-1H-pyrrolo[2,3-b]pyridine-3-sulfonamide;
N-[2-(2,2-difluoroethoxy)-4-methoxy-pyrimidin-5-yl]-6-(trifluoromethyl)-1H-indole-3-sulfonamide;
6-chloro-N-[6-(2,2-difluoroethyl)-5-methoxy-1,2,4-triazin-3-yl]-1H-indole-3-sulfonamide; and
6-chloro-N-(5-methoxy-6-methyl-1,2,4-triazin-3-yl)-1H-indole-3-sulfonamide;
and pharmaceutically acceptable salts thereof.

Preferred examples of compounds of formula I as described herein are selected from
6-chloro-N-[2-(difluoromethoxy)-4-methoxy-pyrimidin-5-yl]-1H-indole-3-sulfonamide;
6-chloro-N-[2-(2,2-difluoroethoxy)-4-(difluoromethoxy)pyrimidin-5-yl]-1H-indole-3-sulfonamide;
6-chloro-N-(2-(2,2-difluoroethoxy)-4-methoxypyrimidin-5-yl)-1H-indole-3-sulfonamide;
6-bromo-N-[2-(2,2-difluoroethoxy)-4-methoxy-pyrimidin-5-yl]-1H-indole-3-sulfonamide;
6-bromo-N-[2-(2,2-difluoroethoxy)-4-methoxy-pyrimidin-5-yl]-1H-pyrrolo[2,3-b]pyridine-3-sulfonamide:
and pharmaceutically acceptable salts thereof.

Processes for the manufacture of compounds of formula I as described herein are an object of the invention.

The present compounds of formula I and their pharmaceutically acceptable salts can be prepared by methods known in the art, for example, by processes described below, which process comprises reacting a compound of formula III with a compound of formula II in the presence of a base selected from N-ethyldiisopropylamine, pyridine, potassium phosphate or sodium hydride to provide a compound of formula I,

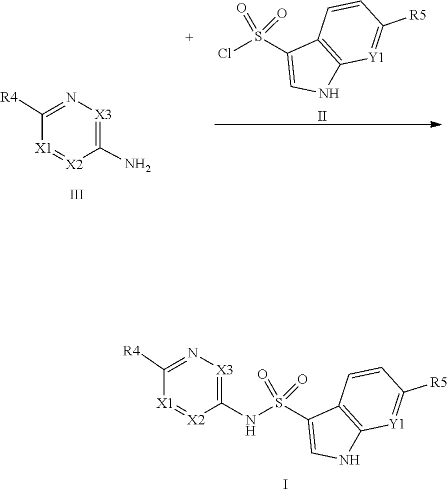

wherein R⁴, R⁵, X₁, X₂, X₃ and Y₁ are as described above.

### General Synthetic Schemes

The compounds of formula I may be prepared in accordance with the process variant described above and with the following scheme 1. The starting materials are commercially available or may be prepared in accordance with known methods.

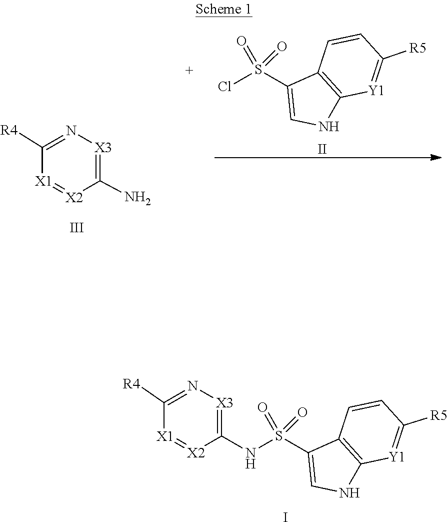

Compounds of general formula I can be prepared by reacting sulfonylchloride II with amines III in the presence of a base like N-ethyldiisopropylamine, pyridine, potassium phosphate or sodium hydride. The starting materials are commercially available or may be prepared in accordance with known methods.

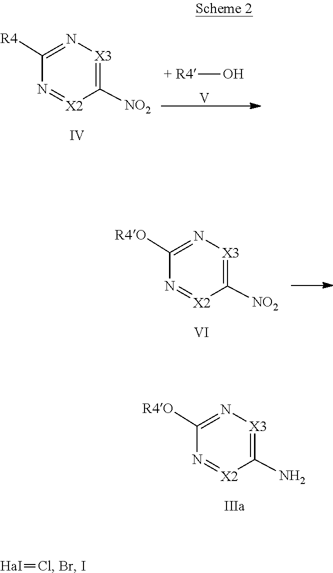

For example, amines Illa can be synthesized by reaction of compounds IV with an alcohol V and a base such as cesium carbonate or potassium carbonate or the like to give compounds VI. Those intermediates can be transformed into compounds IIIa by using a suitable reducing reagent such as hydrogen in combination with a catalyst (palladium on charcoal) or a metal such as iron in presence of an acid.

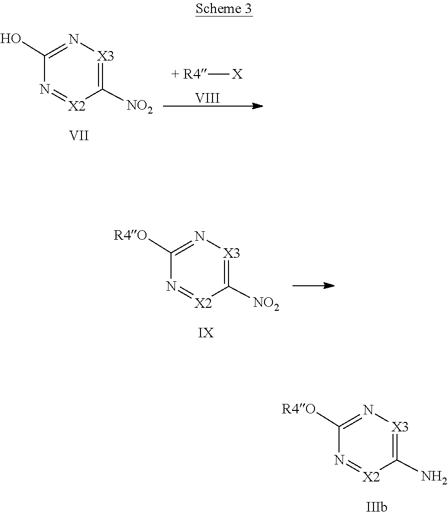

Furthermore, amines IIIb can be synthesized by reaction of compounds VII with an alkylation reagent VIII and a base such as sodium hydride to give compounds IX. Those intermediates can be transformed into compounds IIIb by using a suitable reducing reagent such as hydrogen in combination with a catalyst (palladium on charcoal) or a metal such as iron in presence of an acid.

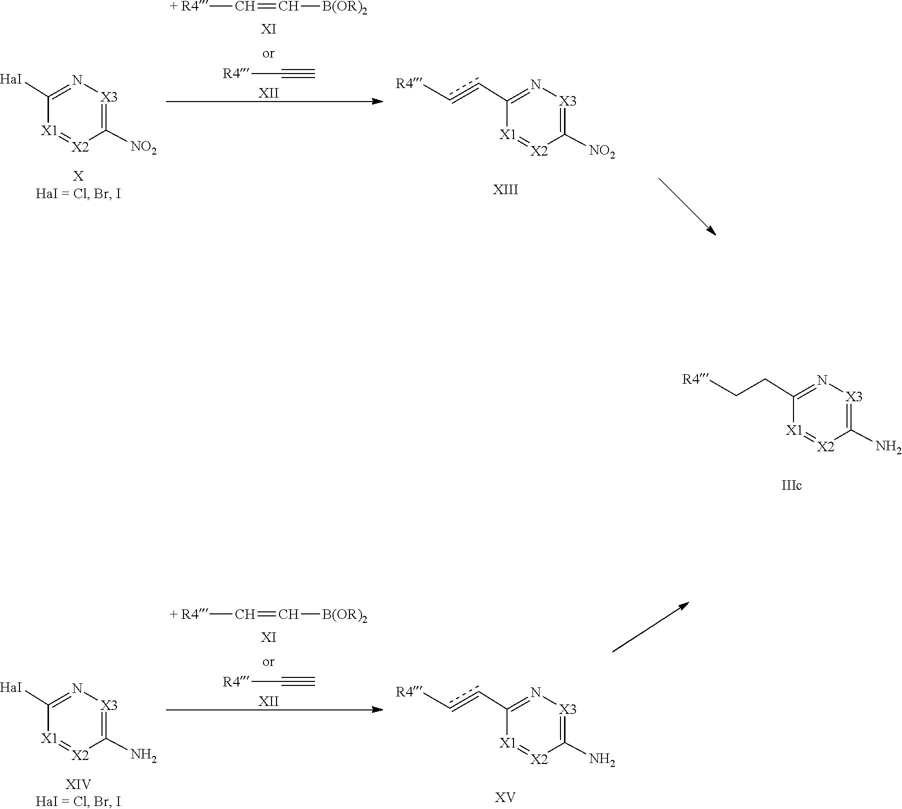

Amines IIIc can be prepared by reaction of compounds X or compounds XIV with an alkenylborane derivative XI or an acetylene XII and a suitable catalyst such as palladium complex or a copper catalyst to give compounds XIII or XV respectively. Those intermediates can be transformed into compounds IIIc by using a suitable reducing reagent such as hydrogen in combination with a catalyst such as palladium on charcoal.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week. It will, however, be clear that the upper or lower limit given herein can be exceeded when this is shown to be indicated.

The invention also relates in particular to:
A compound of formula I for use as therapeutically active substance;
A compound of formula I for use in the treatment of a disease modulated by GPR17;
Likewise an object of the present invention is a pharmaceutical composition comprising a compound according to formula I as described herein and a therapeutically inert carrier.

The use of a compound of formula I for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the treatment or prophylaxis of multiple sclerosis, Alzheimer's disease, Parkinson's disease, or Huntington's disease.

A particular embodiment of the invention is the use of a compound of formula I for the treatment or prophylaxis of multiple sclerosis.

The use of a compound of formula I for the preparation of a medicament for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the preparation of a medicament for the treatment or prophylaxis of multiple sclerosis, Alzheimer's disease, Parkinson's disease, or Huntington's disease.

A particular embodiment of the invention is the use of a compound of formula I for the preparation of a medicament for the treatment or prophylaxis of multiple sclerosis.

A compound according to formula I for use in the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is a compound of formula I for use in the treatment or prophylaxis of multiple sclerosis, Alzheimer's disease, Parkinson's disease, or Huntington's disease.

A particular embodiment of the invention is a compound according to formula I for use in the treatment or prophylaxis of multiple sclerosis.

A method for the treatment or propylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity, which method comprises administering an effective amount of a compound of formula I to a patient in need thereof.

An embodiment of the present invention is a method for the treatment or prophylaxis of multiple sclerosis, Alzheimer's disease, Parkinson's disease, or Huntington's disease, which method comprises administering an effective amount of a compound of formula I to a patient in need thereof.

A particular embodiment of the invention is a method for the treatment or prophylaxis of multiple sclerosis, which method comprises administering an effective amount of a compound of formula I to a patient in need thereof.

Also an embodiment of the present invention provides compounds of formula I as described herein, when manufactured according to any one of the described processes.

### Assay Procedures

GPR17 cAMP Assay Protocol:

CHO-K1 cells stably expressing vector containing untagged human GPR17 short isoform (Roche) were cultured at 37° C./5% CO2 in DMEM (Dulbecco's Modified Eagle Medium): F-12 (1:1) supplemented with 10% foetal bovine serum and 400 μg/ml Geneticin.

Changes in intracellular cyclic adenosine monophosphate (cAMP) levels were quantified using the Nano-TRF Detection Assay kit (Roche Diagnostics, Cat. No. 05214386001). This assay allows for direct cAMP quantification in a homogeneous solution. cAMP is detected based on time-resolved fluorescence energy transfer (TR-FRET) and competitive binding of ruthenylated cAMP and endogenous cAMP to an anti-cAMP monoclonal antibody labeled with AlexaFluor-700. The Ruthenium complex serves as the FRET donor and transfers energy to AlexaFluor-700. The FRET signal is inversely proportional to the CAMP concentration.

CHO-GPR17S cells were detached with Accutase and resuspended in assay buffer consisting of Hank's Balanced Salt Solution (HBSS), 10 mM HEPES (4-(2-hydroxyethyl) piperazine-1-ethanesulfonic acid solution) and 0.1% bovine serum albumin (pH 7.4). The cells were seeded in black 384-well plates (Corning) at a density of 10'000 cells/20 μl assay buffer until the addition of compounds.

Test antagonist compounds were serially diluted in dimethyl sulfoxide (DMSO) and spotted in 384-well plates. The compounds were then diluted in HBSS buffer supplemented with an EC80 concentration of MDL29,951 (3-(2-Carboxy-4,6-dichloroindol-3-yl) propionic acid) (GPR17 agonist) plus 3-Isobutyl-1-methylxanthine (IBMX) (0.5 mM final concentration) and added to the cells at room temperature. Forskolin (15 μM final concentration) was added 5 minutes after the test compounds and the cells were incubated at room temperature for 30 minutes. The assay was stopped by adding cAMP detection mix (containing detergents for cell lysis) for 90 minutes at room temperature.

Cellular cAMP was measured using a Paradigm reader (Molecular Devices). The raw data was used to calculate the FRET signal based on the assay's P-factor as per cAMP kit instructions. The data was normalized to the maximal activity of a reference antagonist and dose response curves were fitted to the percent activity of the test compounds using a sigmoidal dose response model (Genedata Screener).

Results in the hGPR17 cAMP assay are provided for compounds of formula I in Table 1

TABLE 1

| | hGPR17 cAMP |
|---|---|
| Example number | IC50 [μM] |
| 1 | 0.772 |
| 2 | 0.637 |
| 3 | 0.159 |
| 4 | 0.460 |
| 5 | 0.174 |
| 6 | 1.19 |
| 7 | 0.727 |
| 8 | 0.121 |
| 9 | 0.053 |
| 10 | 0.167 |
| 11 | 0.347 |
| 12 | 0.908 |
| 13 | 5.220 |

### Microsome Clearance Assay Protocol:

Incubations at a test compound of 1 mM in microsomes (0.5 mg/mL) plus cofactor NADPH are performed in 96 well plates at 37° C. on a TECAN (Tecan Group Ltd, Switzerland) automated liquid handling system. The final concentration of the test compound in the incubation is 1 microM. After a 10 minutes preincubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standards. Samples are then cooled and centrifuged before analysis of the supernatant by LC-MS/MS.

Reference examples RE-A and RE-B have been prepared as described herein.

Reference compounds were tested against exemplified compounds for their Microsome Clearance to evaluate metabolite stability. The results are shown in Table 2 below.

TABLE 2

| | Microsomal |
|---|---|
| | Clearance |
| Compound | human |
| 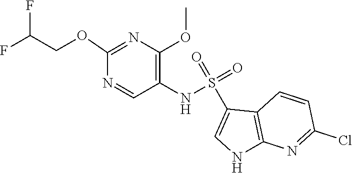 [SMILES: COc1nc(OCC(F)F)ncc1NS(=O)(=O)c1c[nH]c2nc(Cl)ccc12] | 10 μL/min/mg protein |
| Example 4 | |
| 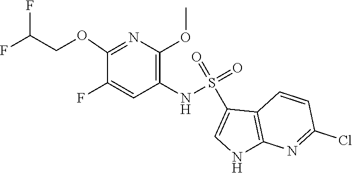 [SMILES: COc1nc(OCC(F)F)c(F)cc1NS(=O)(=O)c1c[nH]c2nc(Cl)ccc12] | 41 μL/min/mg protein |
| RE-A | |
| 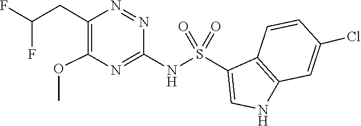 [SMILES: COc1nc(NS(=O)(=O)c2c[nH]c3cc(Cl)ccc23)nnc1CC(F)F] | 10 μL/min/mg protein |
| Example 12 | |
| 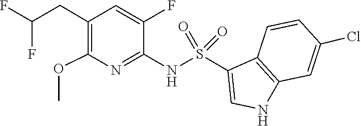 [SMILES: COc1nc(NS(=O)(=O)c2c[nH]c3cc(Cl)ccc23)c(F)cc1CC(F)F] | 35 μL/min/mg protein |
| RE-B | |

The invention will now be illustrated by the following examples which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

All examples and intermediates were prepared under nitrogen atmosphere if not specified otherwise.

### Intermediates A

### Intermediate A1: 6-chloro-1H-indole-3-sulfonyl chloride

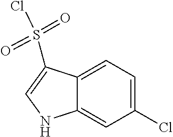 [SMILES: O=S(=O)(Cl)c1c[nH]c2cc(Cl)ccc12]

Intermediate A1 is commercial (CAS 1216060-79-5)

### Intermediate A2: 6-bromo-1H-indole-3-sulfonyl chloride

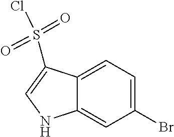 [SMILES: O=S(=O)(Cl)c1c[nH]c2cc(Br)ccc12]

Intermediate A2 is commercial (CAS 2137914-35-1)

### Intermediate A3: 6-bromo-1H-pyrrolo[2,3-b]pyridine-3-sulfonyl chloride

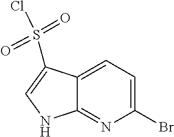 [SMILES: O=S(=O)(Cl)c1c[nH]c2nc(Br)ccc12]

Intermediate A3 is known (CAS 2231234-27-6) and was synthesized according to WO2018/122232.

### Intermediate A4: 6-chloro-1H-pyrrolo[2,3-b]pyridine-3-sulfonyl chloride

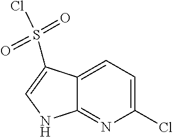 [SMILES: O=S(=O)(Cl)c1c[nH]c2nc(Cl)ccc12]

Intermediate A4 is known (CAS 2231234-21-0) and was synthesized according to WO2018/122232.

### Intermediate A5: 6-(trifluoromethyl)-1H-indole-3-sulfonyl chloride

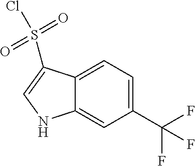 [SMILES: O=S(=O)(Cl)c1c[nH]c2cc(C(F)(F)F)ccc12]

Intermediate A5 is known (CAS 1784173-91-6) and was synthesized according to WO2018/122232.

### Intermediates B

### Intermediate B1: 4-methoxy-2-propyl-pyrimidin-5-amine

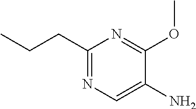

### Step 1: 2-iodo-4-methoxy-5-nitro-pyrimidine

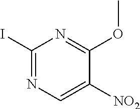

In a 10 ml glass tube under argon, 2-chloro-4-methoxy-5-nitro-pyrimidine (CAS 282102-07-2, 380 mg, 2.0 mmol) was dissolved in acetonitrile, extra dry (4.2 ml) and trimethyliodosilane (481 mg, 361 ul, 2.41 mmol) was added at room temperature. The reaction mixture was stirred 30 min at room temperature and 1 h at 40° C. After cooling to room temperature ethyl acetate (10 ml) was added. The mixture was extracted with NaHCO3 solution (5%), sodium thiosulfate solution (1%) and saturated sodium chloride solution. The combined organic layers were dried with MgSO4, filtered and solvent was removed under vacuo. The residue was purified by flash chromatography (silica gel, 0 to 20% n-heptane in ethyl acetate) to afford 2-iodo-4-methoxy-5-nitro-pyrimidine (310 mg, 44% yield) as light yellow viscous oil. MS m/z: 282.0 [M+H]⁺, ESI pos.

### Step 2: (2-iodo-4-methoxy-pyrimidin-5-yl)amine

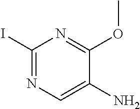

A suspension of 2-iodo-4-methoxy-5-nitro-pyrimidine (205 mg, 0.730 mmol) and iron (326 mg, 5.84 mmol) in acetic acid (6 ml) was stirred at room temperature for 2 h. The reaction mixture was filtered through a pad of Celite and the filtrate was concentrated in vacuo. The residue was poured into saturated NaHCO3 solution and extracted twice with ethyl acetate. The combined organic layers were dried over Na2SO4, filtered and concentrated in vacuo. The residue was purified by chromatography (silica gel, 0 to 20% n-heptane in ethyl acetate) to afford (2-iodo-4-methoxy-pyrimidin-5-yl)amine (157 mg, 73.8%) as white solid. MS m/z: 252.0 [M+H]⁺, ESI pos.

### Step 3: (4-methoxy-2-prop-1-ynyl-pyrimidin-5-yl)amine

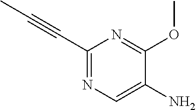

To a solution of prop-1-yne (1M in dimethylformamide, 1.23 ml, 1.23 mmol) was added (2-iodo-4-methoxy-pyrimidin-5-yl)amine (154 mg, 0.613 mmol), triethylamine (186 mg, 256 ul, 1.84 mmol), bis(triphenylphosphine)palladium(II) chloride (30 mg, 0.043 mmol) and copper (I) iodide (6 mg, 0.03 mmol). The solution was stirred at 40° C. for 4 h, then the reaction mixture was diluted with ethyl acetate and extracted with 5% NaHCO3 solution, water and saturated NaCl solution successively. The organic phase was dried with MgSO4, filtered and the solvent was removed under vacuo. The residue was purified by chromatography (silica gel, 0 to 100% n-heptane in ethyl acetate) to afford (4-methoxy-2-prop-1-ynyl-pyrimidin-5-yl)amine (57 mg, 54%) as light yellow solid. MS m/z: 164.1 [M+H]⁺, ESI pos.

### Step 4: 4-methoxy-2-propyl-pyrimidin-5-amine

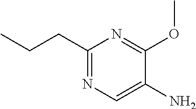

In a 10 ml glass tube under argon, (4-methoxy-2-prop-1-ynyl-pyrimidin-5-yl)amine (55 mg, 0.337 mmol) was dissolved in ethyl acetate (3 ml) and palladium on charcoal 10% (54 mg) was added. After flashing with hydrogen three times a balloon filled with hydrogen gas was applied and the mixture was stirred for 5 h, then filtered through Celite and concentrated to get the title compound (53 mg, 85% yield) as colorless viscous oil. MS m/z: 168.1 [M+H]⁺, ESI pos.

### Intermediate B2: 2-ethyl-4-methoxy-pyrimidin-5-amine

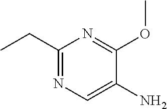

A solution of 2-chloro-4-methoxy-5-nitro-pyrimidine (CAS 282102-07-2, 160 mg, 0.844 mmol), 4,4,5,5-tetramethyl-2-vinyl-1,3,2-dioxaborolane (143 mg, 159 ul, 0.928 mmol) and tripotassium phosphate (538 mg, 2.53 mmol) in 1,4-dioxane (6.4 ml) and water (1.6 ml) was degassed under Ar. Then 1,1′-bis(diphenylphosphino)ferrocenedichloropalladium(II) dichloromethane adduct (69 mg, 0.084 mmol) was added and the reaction mixture was heated for 15 min at 80° C. The mixture was diluted with dichloromethane (20 ml) and filtered on a pad of Celite. Water was added, the aqueous layer was extracted with dichloromethane and the combined organic layers were concentrated. The residue was dissolved in methanol (3 ml) and palladium on charcoal (10%, 65 mg) was added under a flow of argon. A balloon filled with hydrogen gas was applied and the mixture was stirred for 2 h, then filtered through Celite and concentrated. The residue was purified by preparative HPLC (column Gemini NX, 12 nm, 5 um, 100×30 mm, solvent: acetonitrile/water+0.1% trimethylamine) to the title compound (5 mg, 5% yield) as a light brown oil. MS m/z: 154.1 [M+H]⁺, ESI pos.

### Intermediate B3: 2-(difluoromethoxy)-4-methoxy-pyrimidin-5-amine

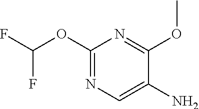

### Step 1: 4-methoxy-5-nitro-pyrimidin-2-ol

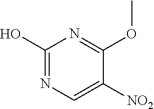

2-Chloro-4-methoxy-5-nitro-pyrimidine (CAS 282102-07-2, 150 mg, 0.79 mmol) was dissolved in ethanol (5 ml). Then a solution of sodium acetate (136 mg, 1.66 mmol) in water (1 ml) and acetic acid (475 mg, 453 ul, 7.9 mmol) were added and the mixture was stirred for 7 h at 80° C.

The mixture was evaporated to dryness and used crude for the next step. Yellow solid, MS m/z: 172.1 [M+H]⁺, ESI pos.

### Step 2: 2-(difluoromethoxy)-4-methoxy-5-nitro-pyrimidine

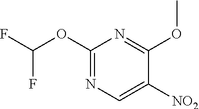

To a solution of 4-methoxy-5-nitro-pyrimidin-2-ol (230 mg, 1.08 mmol) in acetonitrile (5 ml) was added sodium hydride (60%, 151 mg, 3.76 mmol). After stirring for 10 min 2-(fluorosulfonyl)difluoroacetic acid (383 mg, 223 ul, 2.15 mmol) was added and the mixture was stirred for 2.5 h at room temperature. Because the reaction was not complete, more sodium hydride (60%, 151 mg, 3.76 mmol) and 2-(fluorosulfonyl)difluoroacetic acid (383 mg, 223 ul, 2.15 mmol) was added. The reaction mixture was quenched with water at 0° C. and extracted with dichloromethane. The aqueous layer was washed with dichloromethane twice. The combined organic layers were dried using Na2SO4 and evaporated to dryness. The residue was purified by chromatography (silica gel, 0 to 30% n-heptane in ethyl acetate) to afford 2-(difluoromethoxy)-4-methoxy-5-nitro-pyrimidine as colourless oil. MS m/z: 222.1 [M+H]⁺, ESI pos.

### Step 3: 2-(difluoromethoxy)-4-methoxy-pyrimidin-5-amine

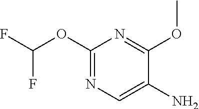

To a solution of 2-(difluoromethoxy)-4-methoxy-5-nitro-pyrimidine (20 mg, 0.09 mmol) in methanol (1 ml)palladium on charcoal 10% (10 mg) was added. A balloon filled with hydrogen gas was applied and the mixture was stirred for 5 h, then filtered over a syringe filter and concentrated to get the title compound (19 mg, 100% yield), MS m/z: 192.1 [M+H]⁺, ESI pos.

### Intermediate B4: 2-(2-fluoroethoxy)-4-methoxy-pyrimidin-5-amine

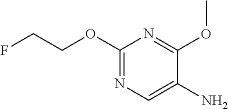

### Step 1: 2-(2-fluoroethoxy)-4-methoxy-5-nitropyrimidine

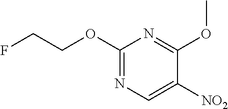

To a solution of 2-chloro-4-methoxy-5-nitropyrimidine (CAS 282102-07-2, 200 mg, 1.06 mmol) in acetonitrile (15 ml) under nitrogen at room temperature, were added 2-fluoroethan-1-ol (101 mg, 92 ul, 1.58 mmol) and cesium carbonate (694 mg, 2.11 mmol). The mixture was stirred at room temperature for 4 h. The reaction mixture was filtered and the filtrate was concentrated in vacuo. The residue was purified by flash chromatography (silica gel, 0% to 30% ethyl acetate in n-heptane) to provide 2-(2-fluoroethoxy)-4-methoxy-5-nitropyrimidine (50 mg, 22% yield) as a light yellow oil. MS m/z: 218.1 [M+H]⁺, ESI pos.

### Step 2: 2-(2-fluoroethoxy)-4-methoxy-pyrimidin-5-amine

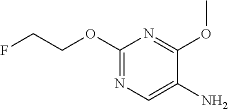

To a solution of 2-(2-fluoroethoxy)-4-methoxy-5-nitropyrimidine (50 mg, 0.23 mmol) in ethyl acetate (1.4 ml) under argon at room temperature, was added palladium on charcoal 10% (25 mg). The reaction mixture was stirred under hydrogen atmosphere using a balloon for 18 h. The reaction mixture was purged with argon, filtered through a Sartorius filter and concentrated in vacuo. The solid was triturated with diethyl ether three times and then dried to provide the title compound (26 mg, 57% yield) as a light brown solid. MS m/z: 188.1 [M+H]⁺, ESI pos.

### Intermediate B5: 2-(2,2-difluoroethoxy)-4-methoxy-pyrimidin-5-amine

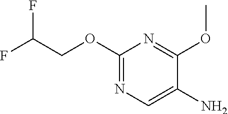

The title compound was prepared in analogy to Intermediate B5 from 2,2-difluoroethan-1-ol instead of 2-fluoroethan-1-ol as light brown solid. MS (ESI) m/z: 206.3 [M+H]⁺.

### Intermediate B6: 2-(2,2-difluoroethoxy)-4-(difluoromethoxy)pyrimidin-5-amine

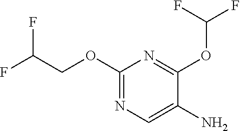 [SMILES: Nc1cnc(OCC(F)F)nc1OC(F)F]

### Step 1: sodium 2-chloro-5-nitropyrimidin-4-olate

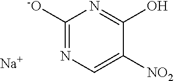

To a light yellow solution of 2,4-dichloro-5-nitropyrimidine (CA 49845-33-2, 2.0 g, 10 mmol) in ethanol (30 ml) under nitrogen at 5° C., was added a solution of sodium acetate (1.69 g, 20.6 mmol) in a mixture of water (5 ml) and glacial acetic acid (2 ml). The reaction mixture was stirred at 5° C. for 3 h. The resulting precipitate was filtered and rinsed with aqueous ethanol. The resulting solid was dried in vacuo to provide sodium 2-chloro-5-nitropyrimidin-4-olate (1.91 g, 97% yield) as a white solid. The compound was used directly without further purification. MS m/z: 174.0 [M−H]⁻, ESI neg.

### Step 2: 2-(2,2-difluoroethoxy)-5-nitropyrimidin-4-ol

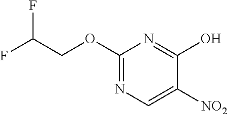 [SMILES: O=[N+]([O-])c1cnc(OCC(F)F)nc1O]

To a suspension of sodium 2-chloro-5-nitropyrimidin-4-olate in acetonitrile (42 ml) under nitrogen at room temperature, were added 2,2-difluoroethan-1-ol (1.28 g, 991 ul, 15.2 mmol) and cesium carbonate (6.63 g, 20.3 mmol). The mixture was stirred at room temperature for 15 h. The reaction mixture was filtered. The mother liquor was quenched with water (100 ml) and acidified with hydrochloric acid (5N) until pH 1. Ethyl acetate (70 ml) was added and both layers were separated. The aqueous one was extracted twice with ethyl acetate. The combined organic layers were dried over Na2SO4 and evaporated to get 110 mg of product. The filter cake was suspended in water, cooled to 0° C. and carefully acidified with hydrochloric acid (5N) until pH 1-2. Ethyl acetate was added and both layers were separated. The aqueous one was extracted twice with ethyl acetate. The combined organic layers, together with the 110 mg product, were dried over Na2SO4 and evaporated to provide 2-(2,2-difluoroethoxy)-5-nitropyrimidin-4-ol (821 mg, 73% yield) as a yellow solid. MS m/z: 222.0 [M+H]⁺, ESI pos

### Step 3: 5-amino-2-(2,2-difluoroethoxy)pyrimidin-4-ol

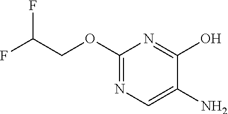 [SMILES: Nc1cnc(OCC(F)F)nc1O]

To a solution of 2-(2,2-difluoroethoxy)-5-nitro-pyrimidin-4-ol (146 mg, 0.660 mmol) in ethyl acetate (7 ml) was added palladium on charcoal 10% (70 mg). The mixture was stirred under hydrogen atmosphere at room temperature for 1 h. The reaction mixture was filtered through a pad of Celite and washed with ethyl acetate. The filtrate was concentrated in vacuo affording 5-amino-2-(2,2-difluoroethoxy)pyrimidin-4-ol (94 mg, 75% yield) as off-white solid. MS m/z: 192.2 [M+H]⁺, ESI pos.

### Step 4: 2-(2,2-difluoroethoxy)-4-(difluoromethoxy)pyrimidin-5-amine

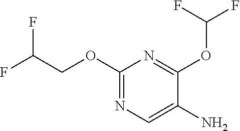 [SMILES: Nc1cnc(OCC(F)F)nc1OC(F)F]

A suspension of 5-amino-2-(2,2-difluoroethoxy)pyrimidin-4-ol (93 mg, 0.487 mmol) and potassium carbonate (204 mg, 1.46 mmol) in N-methyl-2-pyrrolidinone (2.5 ml) was heated to 80° C. A solution of sodium chlorodifluoroacetate (155 mg, 0.973 mmol) in N-methyl-2-pyrrolidinone (0.8 ml) was dropwise added. The reaction mixture was stirred at 80° C. for 30 min. The reaction mixture was poured into water and extracted twice with EtOAc. The organic layers were washed twice with brine, dried over Na2SO4, filtered and concentrated in vacuo. The crude material was purified by flash chromatography (silica gel, 0% to 100% ethyl acetate in heptane) to afford the title compound (24 mg, 10% yield) as light yellow viscous oil. MS m/z: 242.2 [M+H]⁺, ESI pos.

### Intermediate B7: 2,4-dichloropyrimidin-5-amine

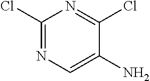

Intermediate B7 is commercial (CAS 5177-27-5)

### Intermediate B8: 6-(2,2-difluoroethyl)-5-methoxy-1,2,4-triazin-3-amine

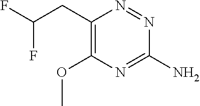

### Step 1: 6-chloro-5-methoxy-N,N-bis[(4-methoxyphenyl)methyl]-1,2,4-triazin-3-amine

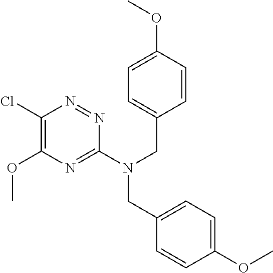 [SMILES: COc1ccc(CN(Cc2ccc(OC)cc2)c2nnc(Cl)c(OC)n2)cc1]

In a 100 ml three-necked flask, 3,5,6-trichloro-1,2,4-triazine (CAS 873-41-6, 1.6 g, 8.68 mmol) was dissolved in tetrahydrofuran (25 ml) and the solution was cooled to 0° C. Sodium methoxide (25% in MeOH, 1.87 g, 2.0 mL, 8.68 mmol) was added. The ice bath was removed and the reaction mixture was stirred at room temperature for 1 h. Then it was cooled to 0° C., N,N-diisopropylethylamine (1.68 g, 2.27 mL, 13.01 mmol) and bis(p-methoxyphenyl)amine (2.23 g, 8.68 mmol) were added. The reaction mixture was stirred 1 h at room temperature, then at 50° C. overnight. The reaction mixture was poured into water and extracted with ethyl acetate twice. The combined organic layers were washed with brine, dried over sodium sulfate, filtered and concentrated in vacuo. The crude material was purified by flash chromatography (silica gel, 30% ethyl acetate in heptane) to afford 6-chloro-5-methoxy-N,N-bis[(4-methoxyphenyl)methyl]-1,2,4-triazin-3-amine (1.25 g, 36% yield) as light yellow liquid. MS m/z: 401.3 [M+H]⁺, ESI pos.

### Step 2: 6-(2-ethoxyvinyl)-5-methoxy-N,N-bis[(4-methoxyphenyl)methyl]-1,2,4-triazin-3-amine

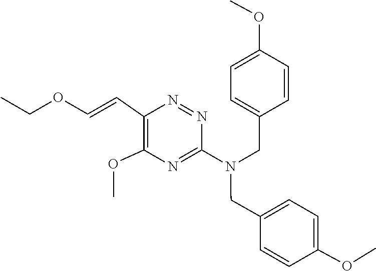 [SMILES: CCO/C=C/c1nnc(N(Cc2ccc(OC)cc2)Cc2ccc(OC)cc2)nc1OC]

To a solution of 6-chloro-5-methoxy-N,N-bis[(4-methoxyphenyl)methyl]-1,2,4-triazin-3-amine (1 g, 2.49 mmol) in 1,4-dioxane (25 ml), 2-[(E)-2-ethoxyvinyl]-4,4,5,5-tetramethyl-1,3,2-dioxaborolane (593 mg, 634 ul, 3.0 mmol), sodium carbonate (2M solution in water, 3.7 ml, 7.48 mmol) and 1,1′-bis(diphenylphosphino)ferrocene-palladium(II) dichloride dichloromethane complex (206 mg, 0.25 mmol) were added under argon. The reaction mixture was stirred at 90° C. for 4 h. After cooling to room temperature, the reaction mixture was poured into water and extracted with ethyl acetate twice. The combined organic layers were washed with brine, dried over sodium sulfate, filtered and concentrated in vacuo. The residue was purified by flash chromatography (silica gel, 0% to 30% ethyl acetate in heptane) to afford 6-(2-ethoxyvinyl)-5-methoxy-N,N-bis[(4-methoxyphenyl)methyl]-1,2,4-triazin-3-amine (432 mg, 40% yield) as colorless liquid. MS m/z: 437.4 [M+H]⁺, ESI pos.

### Step 3: 6-(2,2-difluoroethyl)-5-methoxy-N,N-bis[(4-methoxyphenyl)methyl]-1,2,4-triazin-3-amine

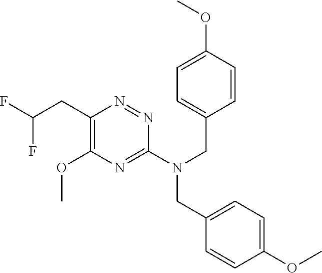 [SMILES: COc1ccc(CN(Cc2ccc(OC)cc2)c2nnc(CC(F)F)c(OC)n2)cc1]

In a 25 ml round-bottomed flask, 6-(2-ethoxyvinyl)-5-methoxy-N,N-bis[(4-methoxyphenyl)methyl]-1,2,4-triazin-3-amine (330 mg, 0.756 mmol) was dissolved in formic acid (5 ml) and the mixture was stirred at 60° C. for 1.5 h. After cooling to room temperature, the reaction mixture was poured into ice-water and extracted with dichloromethane twice. The combined organic layers were washed with sodium bicarbonate solution, dried over sodium sulfate, filtered and concentrated in vacuo. The residue was dissolved in dichloromethane (16.5 ml) and cooled to 0° C., then [bis(2-methoxyethyl)amino]sulfur trifluoride (Deoxofluor, 2.7M in toluene, 672 mg, 560 ul, 1.51 mmol) was added. The reaction mixture was further stirred at 0° C. for 1.5 h, then poured into ice-cold sodium bicarbonate solution and extracted with dichloromethane twice. The combined organic layers were washed with brine, dried over sodium sulfate, filtered and concentrated in vacuo. The residue was purified by flash chromatography (silica gel, 0% to 50% ethyl acetate in heptane) to afford 6-(2,2-difluoroethyl)-5-methoxy-N,N-bis[(4-methoxyphenyl)methyl]-1,2,4-triazin-3-amine (133 mg, 41% yield) as light brown oil. MS m/z: 431.4 [M+H]⁺, ESI pos.

### Step 4: 6-(2,2-difluoroethyl)-5-methoxy-1,2,4-triazin-3-amine

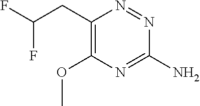

A mixture of 6-(2,2-difluoroethyl)-5-methoxy-N,N-bis[(4-methoxyphenyl)methyl]-1,2,4-triazin-3-amine (70 mg, 0.154 mmol) and trifluoroacetic acid (1.59 g, 1.07 ml, 13.9 mmol) was stirred at 70° C. for 3 days. The reaction mixture was concentrated in vacuo and the residue was dissolved in dichloromethane, poured into saturated sodium bicarbonate solution and extracted twice with ethyl acetate. The combined organic layers were washed with brine, dried over sodium sulfate and concentrated in vacuo. The crude material was purified by flash chromatography (silica gel, 30 to 100% ethyl acetate in heptane) to afford the title compound (28 mg, 98% yield) as off-white solid. MS m/z: 191.1 [M+H]⁺, ESI pos.

### Intermediate B9: 5-methoxy-6-methyl-1,2,4-triazin-3-amine

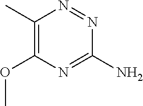

3-Amino-6-methyl-4,5-dihydro-1,2,4-triazin-5-one (CAS 1004-04-2, 100 mg, 0.793 mmol) was added at 22° C. to phosphorus oxychloride (3.65 g, 2.22 ml, 23.8 mmol). The mixture was stirred at 80° C. for 1 h, then concentrated in vacuo. The residue was dissolved in methanol (5 ml) and the solution was added to sodium methoxide (128 mg, 2.38 mmol) and stirred at 22° C. for 1.5 h. The mixture was quenched with aqueous saturated sodium bicarbonate solution (10 ml) and extracted with ethyl acetate (2×10 ml). The organic layers were dried over sodium sulfate, filtered and concentrated in vacuo to the title compound (55 mg, 50% yield) as light yellow solid. MS m/z: 141.1 [M+H]⁺, ESI pos.

### EXAMPLES

### Example 1:6-chloro-N-(4-methoxy-2-propyl-pyrimidin-5-yl)-1H-indole-3-sulfonamide

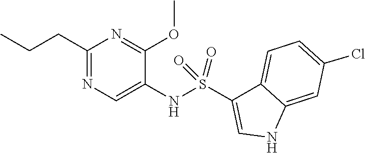 [SMILES: CCCc1ncc(NS(=O)(=O)c2c[nH]c3cc(Cl)ccc23)c(OC)n1]

A mixture of 6-chloro-1H-indole-3-sulfonyl chloride (Intermediate A1, 67 mg, 0.269 mmol) and 4-methoxy-2-propyl-pyrimidin-5-amine (Intermediate B1, 50 mg, 0.269 mmol) in dichloromethane (extra dry, 1.3 ml) was cooled to 2-5° C. N,N-Diisopropylethylamine (45 mg, 61 ul, 0.350 mmol) was added, after 20 min the cooling bath was removed and the reaction mixture was stirred at room temperature for 2 h. The reaction mixture was poured into water and extracted with ethyl acetate. The combined organic layers were washed with brine, dried over sodium sulfate, filtered and concentrated in vacuo. The residue was purified by preparative HPLC (column Gemini NX, 12 nm, 5 um, 100×30 mm, solvent: acetonitrile/water+0.1% HCOOH) to get the title compound (46 mg, 45%) as white solid. MS m/z: 381.2 [M+H]⁺, ESI pos.

The following Examples 2-6 were prepared in analogy to Example 1 by coupling the indicated sulfonylchloride intermediates A and amine intermediates B.

| | | | | | MS |
|---|---|---|---|---|---|
| Ex. | Structure | Name | Int. A | Int. B | (ESI): m/z |
| 2 | 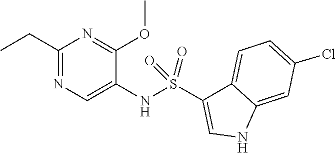 [SMILES: CCc1ncc(NS(=O)(=O)c2c[nH]c3cc(Cl)ccc23)c(OC)n1] | 6-chloro-N-(2-ethyl- 4-methoxy-pyrimi- din-5-yl)-1H-indole- 3-sulfonamide | A1 | B2 | 367.2 [M + H]⁺ |
| 3 | 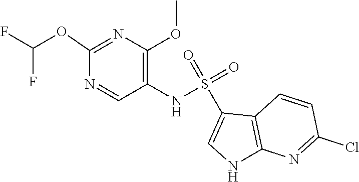 [SMILES: COc1nc(OC(F)F)ncc1NS(=O)(=O)c1c[nH]c2nc(Cl)ccc12] | 6-chloro-N-[2- (difluoromethoxy)- 4-methoxy-pyrimi- din-5-yl]-1H-indole- 3-sulfonamide | A1 | B3 | 405.2 [M + H]⁺ |
| 4 | 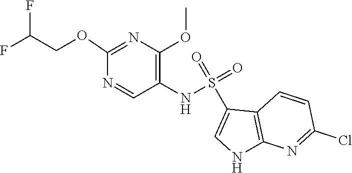 [SMILES: COc1nc(OCC(F)F)ncc1NS(=O)(=O)c1c[nH]c2nc(Cl)ccc12] | 6-chloro-N-[2-(2,2- difluoroethoxy)-4- methoxy-pyrimidin- 5-yl]-1H-pyrrolo [2,3-b]pyridine-3- sulfonamide | A4 | B5 | 420.2 [M + H]⁺ |
| 5 | 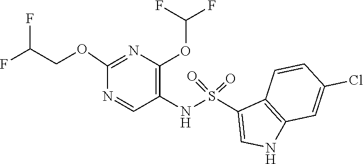 [SMILES: O=S(=O)(Nc1cnc(OCC(F)F)nc1OC(F)F)c1c[nH]c2cc(Cl)ccc12] | 6-chloro-N-[2-(2,2- difluoroethoxy)-4- (difluoromethoxy)- pyrimidin-5-yl]-1H- indole-3-sulfon- amide | A1 | B6 | 455.1 [M + H]⁺ |
| 6 | 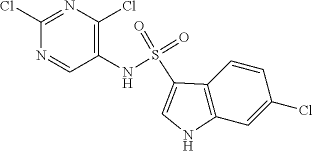 [SMILES: O=S(=O)(Nc1cnc(Cl)nc1Cl)c1c[nH]c2cc(Cl)ccc12] | 6-chloro-N-(2,4- dichloropyrimidin- 5-yl)-1H-indole-3- sulfonamide | A1 | B7 | 379.0 [M + H]⁺ |

### Example 7: 6-chloro-N-(2-(2-fluoroethoxy)-4-methoxypyrimidin-5-yl)-1H-indole-3-sulfonamide

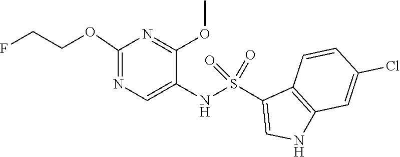 [SMILES: COc1nc(OCCF)ncc1NS(=O)(=O)c1c[nH]c2cc(Cl)ccc12]

To solution of 2-(2-fluoroethoxy)-4-methoxy-pyrimidin-5-amine (Intermediate B4, 25 mg, 0.134 mmol) in pyridine (dry, 0.85 ml) under argon at 0° C., was added portionwise 6-chloro-1H-indole-3-sulfonyl chloride (Intermediate A1, 200 mg, 0.40 mmol), followed by 4-dimethylaminopyridine (1.7 mg, 0.013 mmol). The mixture was stirred at 100° C. for 2.5 h, then it was cooled to room temperature and evaporated. The residue was partitioned between citric acid (1M solution in water) and ethyl acetate. Both layers were separated and the aqueous one was extracted twice with ethyl acetate. The combined organic layers were dried over sodium sulfate and evaporated. The residue was purified with preparative HPLC (column Gemini NX, 12 nm, 5 um, 100×30 mm, solvent: acetonitrile/water+0.1% HCOOH) to provide the title compound (11 mg, 20% yield) as an off-white solid. MS m/z: 399.1 [M−H]⁻, ESI neg.

The following Examples 8-12 were prepared in analogy to Example 7 by coupling the indicated sulfonylchloride intermediates A and amine intermediates B.

| | | | | | MS |
|---|---|---|---|---|---|
| Ex. | Structure | Name | Int. A | Int. B | (ESI): m/z |
| 8 | 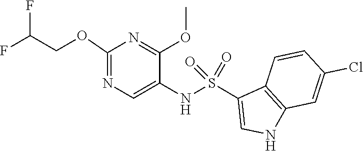 [SMILES: COc1nc(OCC(F)F)ncc1NS(=O)(=O)c1c[nH]c2cc(Cl)ccc12] | 6-chloro-N-(2-(2,2- difluoroethoxy)-4- methoxypyrimidin- 5-yl)-1H-indole-3- sulfonamide | A1 | B5 | 417.1 [M − H]⁻ |
| 9 | 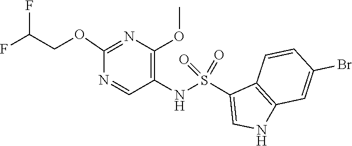 [SMILES: COc1nc(OCC(F)F)ncc1NS(=O)(=O)c1c[nH]c2cc(Br)ccc12] | 6-bromo-N-[2-(2,2- difluoroethoxy)-4- methoxy-pyrimidin- 5-yl]-1H-indole-3- sulfonamide | A2 | B5 | 463.1 [M + H]⁺ |
| 10 | 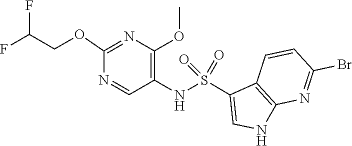 [SMILES: COc1nc(OCC(F)F)ncc1NS(=O)(=O)c1c[nH]c2nc(Br)ccc12] | 6-bromo-N-[2-(2,2- difluoroethoxy)-4- methoxy-pyrimidin- 5-yl]-1H-pyrrolo [2,3-b]pyridine-3- sulfonamide | A3 | B5 | 464.0 [M + H]⁺ |
| 11 | 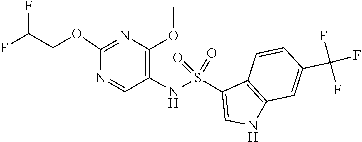 [SMILES: COc1nc(OCC(F)F)ncc1NS(=O)(=O)c1c[nH]c2cc(C(F)(F)F)ccc12] | N-[2-(2,2-difluoro- ethoxy)-4-methoxy- pyrimidin-5-yl]-6- (trifluoromethyl)-1H- indole-3-sulfonamide | A5 | B5 | 451.2 [M − H]⁻ |
| 12 | 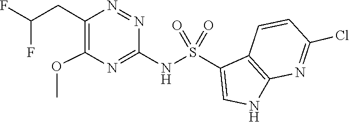 [SMILES: COc1nc(NS(=O)(=O)c2c[nH]c3nc(Cl)ccc23)nnc1CC(F)F] | 6-chloro-N-[6-(2,2- difluoroethyl)-5- methoxy-1,2,4-tria- zin-3-yl]-1H-indole- 3-sulfonamide | A1 | B8 | 404.2 [M + H]⁺ |

### Example 13: 6-chloro-N-(5-methoxy-6-methyl-1,2,4-triazin-3-yl)-1H-indole-3-sulfonamide

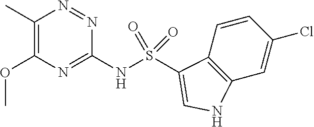 [SMILES: COc1nc(NS(=O)(=O)c2c[nH]c3cc(Cl)ccc23)nnc1C]

A reaction tube was flashed with argon, then filled with 5-methoxy-6-methyl-1,2,4-triazin-3-amine (Intermediate B9, 40 mg, 0.285 mmol) and dry 1,2-dichloroethane (2.9 ml) followed by N-ethyldiisopropylamine (48 mg, 63 ul, 0.371 mmol). The tube was sealed and heated to 70° C. and 6-chloro-1H-indole-3-sulfonyl chloride (Intermediate A1, 78 mg, 0.314 mmol) was added in five portions over a period of 30 min. The mixture was stirred at 70° C. for 1 h, then cooled to room temperature and concentrated in vacuo. The residue was purified by preparative HPLC (column YMC-Triart C18, 12 nm, 5 um, 100×30 mm, solvent: acetonitrile/water+0.1% HCOOH) to give the title compound (14 mg, 14%) as white solid. MS m/z: 354.1 [M+H]⁺, ESI pos.

### Reference Example RE-A: 6-chloro-N-[6-(2,2-difluoroethoxy)-5-fluoro-2-methoxy-3-pyridyl]-1H-pyrrolo[2,3-b]pyridine-3-sulfonamide

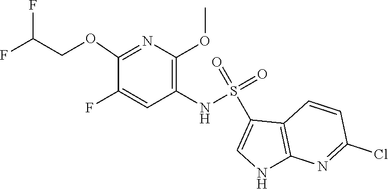 [SMILES: COc1nc(OCC(F)F)c(F)cc1NS(=O)(=O)c1c[nH]c2nc(Cl)ccc12]

RE-A is Examples 1-46 in WO2019243303 and was synthetized following the procedure described therein.

### Reference Example RE-B: 6-chloro-N-[5-(2,2-difluoroethyl)-3-fluoro-6-methoxy-2-pyridyl]-1H-indole-3-sulfonamide

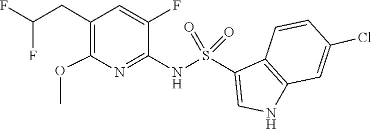 [SMILES: COc1nc(NS(=O)(=O)c2c[nH]c3cc(Cl)ccc23)c(F)cc1CC(F)F]

To a solution of 5-(2,2-difluoroethyl)-3-fluoro-6-methoxy-pyridin-2-amine (CAS 2827059-87-8, 58 mg, 0.281 mmol) in pyridine (1 ml) was added 6-chloro-1H-indole-3-sulfonyl chloride (Intermediate A1, 77 mg, 0.31 mmol) and the reaction mixture was stirred 90 min at room temperature. The reaction mixture was concentrated in vacuo and purified by flash chromatography (silica gel, 0-50% ethyl acetate in heptane) to give the title compound (33 mg, 28%) as white solid. MS m/z: 420.2 [M+H]⁺, ESI pos.

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. Compounds of formula I 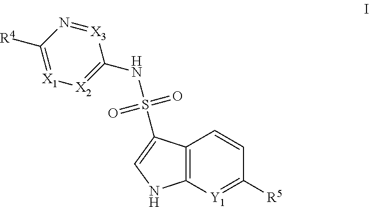
wherein,
R⁴ is alkyl, halo, haloalkyl, or haloalkoxy;
R⁵ is halo or haloalkyl;
X₁ is N, X₂ is CR² and X₃ is CR³, wherein R² is H and R³ is alkoxy, halo, or haloalkoxy, or
X₁ is CR¹, X₂ is N and X₃ is N, wherein R¹ is alkoxy;
Y₁ is CR⁶ or N;
R⁶ is H or halo;
and pharmaceutically acceptable salts thereof.

2. A compound according to claim 1, wherein R⁴ is haloalkoxy.

3. A compound according to claim 1, wherein R⁵ is halo.

4. A compound according to claim 1, wherein R⁶ is H.

5. A compound according to claim 1,
wherein X₁ is N, X₂ is CR², and X₃ is CR³ wherein R² is H and R³ is alkoxy, or haloalkoxy.

6. A compound according to claim 1 wherein
R⁴ is haloalkoxy;
R⁵ is halo;
X₁ is N, X₂ is CR² and X₃ is CR³, wherein R² is H and R³ is alkoxy or haloalkoxy;
Y₁ is CR⁶ or N;
R⁶ is H;
and pharmaceutically acceptable salts thereof.

7. A compound according to claim 1, selected from
6-chloro-N-(4-methoxy-2-propyl-pyrimidin-5-yl)-1H-indole-3-sulfonamide;
6-chloro-N-(2-ethyl-4-methoxy-pyrimidin-5-yl)-1H-indole-3-sulfonamide;
6-chloro-N-[2-(difluoromethoxy)-4-methoxy-pyrimidin-5-yl]-1H-indole-3-sulfonamide;
6-chloro-N-[2-(2,2-difluoroethoxy)-4-methoxy-pyrimidin-5-yl]-1H-pyrrolo[2,3-b]pyridine-3-sulfonamide;
6-chloro-N-[2-(2,2-difluoroethoxy)-4-(difluoromethoxy)pyrimidin-5-yl]-1H-indole-3-sulfonamide;
6-chloro-N-(2,4-dichloropyrimidin-5-yl)-1H-indole-3-sulfonamide;
6-chloro-N-(2-(2-fluoroethoxy)-4-methoxypyrimidin-5-yl)-1H-indole-3-sulfonamide;
6-chloro-N-(2-(2,2-difluoroethoxy)-4-methoxypyrimidin-5-yl)-1H-indole-3-sulfonamide;
6-bromo-N-[2-(2,2-difluoroethoxy)-4-methoxy-pyrimidin-5-yl]-1H-indole-3-sulfonamide;
6-bromo-N-[2-(2,2-difluoroethoxy)-4-methoxy-pyrimidin-5-yl]-1H-pyrrolo[2,3-b]pyridine-3-sulfonamide;
N-[2-(2,2-difluoroethoxy)-4-methoxy-pyrimidin-5-yl]-6-(trifluoromethyl)-1H-indole-3-sulfonamide;
6-chloro-N-[6-(2,2-difluoroethyl)-5-methoxy-1,2,4-triazin-3-yl]-1H-indole-3-sulfonamide;
6-chloro-N-(5-methoxy-6-methyl-1,2,4-triazin-3-yl)-1H-indole-3-sulfonamide;
and pharmaceutically acceptable salts thereof.

8. A compound according to claim 1 any one of claims 1 to 7, selected from
6-chloro-N-[2-(difluoromethoxy)-4-methoxy-pyrimidin-5-yl]-1H-indole-3-sulfonamide;
6-chloro-N-[2-(2,2-difluoroethoxy)-4-(difluoromethoxy)pyrimidin-5-yl]-1H-indole-3-sulfonamide;
6-chloro-N-(2-(2,2-difluoroethoxy)-4-methoxypyrimidin-5-yl)-1H-indole-3-sulfonamide;
6-bromo-N-[2-(2,2-difluoroethoxy)-4-methoxy-pyrimidin-5-yl]-1H-indole-3-sulfonamide;
6-bromo-N-[2-(2,2-difluoroethoxy)-4-methoxy-pyrimidin-5-yl]-1H-pyrrolo[2,3-b]pyridine-3-sulfonamide:
and pharmaceutically acceptable salts thereof.

9. A process to prepare a compound according to claim 1 comprising the reacting a compound of formula III with a compound of formula II in the presence of a base selected from N-ethyldiisopropylamine, pyridine, potassium phosphate or sodium hydride to provide a compound of formula I, 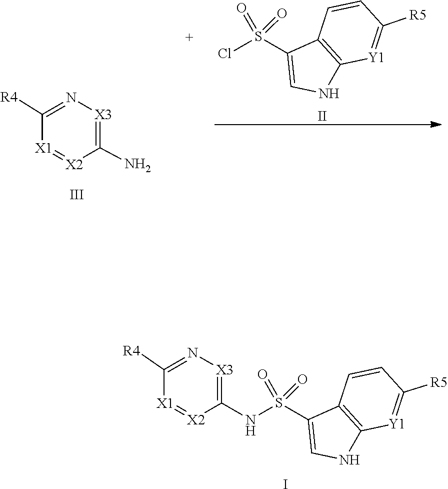
wherein R⁴, R⁵, X₁, X₂, X₃ and Y₁ are as described above.

10. A compound according to claim 1 for use as therapeutically active substance.

11. A compound according to claim 1 for use in the treatment of a disease modulated by GPR17.

12. A pharmaceutical composition comprising a compound according to claim 1 and a therapeutically inert carrier.

13-17. (canceled)

18. A method for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and inflammation in the CNS for instance following encephalitis, primary angiitis, meningitis and obesity, which method comprises administering an effective amount of a compound according to claim 1 to a patient in need thereof.

19. A method for the treatment or prophylaxis of multiple sclerosis, which method comprises administering an effective amount of a compound according to claim 1 to a patient in need thereof.

20-21. (canceled)
